(19) United States
(12) Patent Application Publication
(10) Pub. No.: US 2005/0014200 A1
(43) Pub. Date: Jan. 20, 2005

(54) Autoimmunity to angiotensin at1 receptors in schizophrenia

(76) Inventor: Brian MacVicar, Bristish Columbia (CA)

Correspondence Address: SIM & MCBURNEY, 330 UNIVERSITY AVENUE, 6TH FLOOR, TORONTO, ON, M5G 1R7, CA

(21) Appl. No.: 10/343,457

(22) PCT Filed: Aug. 2, 2001
(86) PCT No.: PCT/CA01/01154

(30) Foreign Application Priority Data
Aug. 2, 2000 (CA) 2,314,811

(51) Int. Cl. G01N 33/53; G01N 33/567; C07K 14/705

(52) U.S. Cl. 435/7.2; 530/350

(57) ABSTRACT

A method of determining whether an individual has schizophrenia or the propensity to develop schizophrenia is provided. The method involves the detection of autoantibodies to the angiotensin AT1 receptor. Diagnostic kits and assays arc also provided within the scope of the invention.

### FIELD OF THE INVENTION

The present invention relates to the field of autoimmnunity, particularly the role of autoantibodies in schizophrenia.

### BACKGROUND OF THE INVENTION

Schizophrenia is a disease in which behaviour is considerably modified. In schizophrenia there is a disorder of thinking processes characterized by delusions, auditory hallucinations, lack of affect, and social withdrawal. The underlying pathology that causes schizophrenia is unknown. However there is circumstantial evidence that there is an immune component to schizophrenia (Ganguli et al., 1993; Kirch, 1993; Ganguli et al., 1994). Furthermore, antibodies against heat shock proteins, which are often observed in autoimmune diseases have been found in schizophrenics (Kilidireas et al., 1992; Schwarz et al., 1999). The autoimmune hypothesis suggests that the immune system aberrantly attacks and modifies neuronal activity in the key brain regions known to be involved in schizophrenia. However, to date no one has identified any proteins that are actually expressed in the brain that are recognized by autoantibodies present in schizophrenics. The identification of autoantibodies has been a key step in determining the autoimmune basis of other diseases. For example the identification of GAD64 as a major protein target for the immune system in diabetes (Baekkeskov et al., 1990) and glutamate receptors in Rasmussen's encephalitis (Rogers et al., 1994; Andrews and McNamara, 1996) made major contributions to understanding the autoimmune basis for these diseases.

Some types of autoimmune diseases are thought to result from the immune system mistakenly attacking normal components of the body such as proteins. This can result from the response of the immune system to infections in which some protein components of the infectious agent are similar to normal protein components of the body's own proteins. This is called molecular mimicry and may trigger autoimmune attacks. In other types of autoimmune diseases, the autoantibodies do not cause tissue damage. In hyperthyroidism, for example, thyroid stimulating autoantibodies stimulate the thyroid stimulating hormone receptor and induce the thyroid to overproduce thyroid hormones.

Schizophrenia is a complex disease for which there is a great need for rapid and easy-to-perform diagnostic tests. The role of autoantibodies in schizophrenia has not been clearly established. However, if autoantibodies alter neuronal function in schizophrenia then they should recognize proteins expressed in the regions of the CNS that are involved in schizophrenia. So far, no such antibodies have been found. Thus there is a need to identify antigens which are expressed in the brain in schizophrenics and which are involved in the disease. Identification of such autoantigens could lead to the development of diagnostic tests.

The current methods for the diagnosis of schizophrenia are time-consuming consuming and not easily adapted to widespread screening. Thus, not all those in need of care receive a timely diagnosis. There remains a large and unmet need for improved diagnostic and screening systems for schizophrenia that are rapid, easy-to-use and inexpensive.

### SUMMARY OF THE INVENTION

As discussed above, there is a need for methods to rapidly screen for schizophrenia and for the propensity to develop schizophrenia. Such screening tests could lead to early diagnosis and more appropriate treatments. In accordance with one aspect, the present invention is directed toward methods and diagnostic kits for the accurate and sensitive detection of autoantibodies to the angiotensin AT1 receptor which are associated with the disease, schizophrenia.

In accordance with a further aspect of the invention, there is provided a method for detecting schizophrenia, the preclinical onset of shizophrenia or the propensisty to develop schizophrenia in a person, said method comprising:
i) contacting a sample containing from the person with an antigen comprising the AT1 receptor or a fragment thereof; and
ii) detecting binding of antibodies in said sample to said antigen; wherein the presence of said binding indicates that said person may have or may be at risk of developing schizophrenia.

In accordance with another aspect of the invention a peptide, comprising all or part of the amino acid sequence of the AT1 receptor, is provided which is recognized by antibodies obtained from schizophrenic patients and recognized only weakly or not at all by control subjects.

In a further aspect of the invention the peptide is used in a kit for the diagnosis of schizophrenia.

In another aspect there is provided a kit for the diagnosis of schizophrenia comprising:
i) the AT1 receptor or a fragment thereof;
ii) positive and negative controls for reactivity to said receptor; and
iii) reagent means for detecting binding of antibody to said receptor.

In yet another aspect, there is provided an assay for the diagnosis of schizophrenia in an individual comprising the steps:
A. obtaining an antibody containing sample from said individual,
B. contacting said sample with an antigen comprising the AT1 receptor or a fragment thereof,
C. determining the level of binding of said sample to said antigen, wherein a level of binding higher than a binding level of a sample from a control is indicative of schizophrenia.

### BRIEF DESCRIPTION OF THE DRAWINGS

The present invention will be described with respect to the drawings in which:

FIG. 1 is a Western blot comparing binding of schizophrenic patient serum sample and control serum samples to glycoproteins prepared from hippocampus.

FIG. 2 illustrates two Western blots, each of which were probed with the commercial anti-AT1 receptor antibodies and with the patient sera;

FIG. 3 is a Western blot indicating antibody binding to immuno-affinity purified AT1 receptor.

### DETAILED DESCRIPTION

As discussed above, there are currently no rapid and quantitative tests for the diagnosis of schiozophrenia. Most diagnoses are made on the basis of extensive testing by specifically trained medical personnel. Seriously ill individuals may experience long waits before being accurately assessed. As with many diseases early intervention often prevents serious situations from developing. A reliable, rapid, and easy-to-use screening assay would help to ensure that those who need help receive it in a timely manner.

While autoantibodies have been identified in other autoimmune diseases and while there have been suspicions that there may be an autoimmune component to schizophrenia, there has not previously been any identification of autoantibodies, found in schizophrenic patients, which recognize a biologically relevant target. In the present invention, shizophrenic patients were found to have autoantibodies which recognize a 64 kDa antigen expressed in the hippocampus of human and rat brains. The same protein was recognized only weakly or not at all by control serum antibodies. This protein was identified as the angiotensin AT1 receptor. The angiotensin AT1 receptor is found in the hippocampus and is known to be associated with a condition of increased drinking called polydipsia. Polydipsia is a common symptom in schizophrenic patients. These results suggest that AT1 receptors and/or antibodies to them may be involved in the pathogenesis of schizophrenia. Surprisingly, autoantobodies to a receptor not previously thought to be involved in schizophrenia were found in greater than 70% of schizophrenic patient samples tested. The control samples recognized the AT1 receptor only weakly or not at all.

In view of this surprising result, it is clearly apparent that the sensitive detection of autoantibodies to AT1 receptor can be used as an indicator of schizophrenic disease. The level of autoantibody may also be indicative of the severity and/or onset of disease. Early detection provides the opportunity for treatments which may forstall serious problems.

Screening assays for autoantibodies may include Western blot analysis, immunoblots, ELISA's, RIA's, FACS analysis and the like. In situ labelling and imaging may also be used. Kits can be developed for liquid phase assays as well as dip-stick type tests. For example, an ELISA kit could include 1) purified AT1 receptor antigen, 2) a detecting, enzyme linked, anti-human immunoglobulin antibody, 3) an enzyme substrate, 4) positive and negative controls. Alternatively, a dip-stick type test, similar to those currently available for HIV, could be developed in which the antigen is immobilized on a strip of nitrocellulose which is reacted with a drop of patient serum followed by reaction with the sequential detecting reagents. It is clearly apparent that various other types of kits designed to detect the presence of autoantibodies can be developed.

Autoantibodies to AT1 may also be used to delineate the role of such antibodies in the pathogenesis or symptomology of schizophrenia. The autoantibodies can be used to determine whether binding of such antibodies to the AT1 receptor affects the receptor associated signaling pathways in a positive or negative manner. It can also be determined whether any such changes in the signaling pathway can be correlated with schizophrenic symptoms.

The autoantibodies of the present invention may be used to develop animal models of schizophrenia based on the interaction of antibodies with the AT1 receptor. Such models may be used to study the pathogenesis of the interaction and to screen novel therapeutic strategies.

Autoantibodies to the AT1 receptor may also be used to identify antigenic epitopes on the AT1 receptor expressed in the brain which are recognized by the autoantibodies. Once the epitopes have been identified, synthetic peptides can be prepared. These peptides can be used as antigens in antibody screening assays and diagnostic kits. These peptides may also be used to develop therapeutic treatments. For example, tolerogenic peptide conjugates, such as peptide-PEG conjugates, could be used to induce tolerance to the antigenic epitopes. Other tolerogenic protocols can also be employed. The identification of the antibody binding epitopes may also be used to develop therapeutic mimetics or analogues which effect the AT1 receptor signaling pathway.

The identification of autoantibodies according to the present invention can also be used to investigate the potential role of cell mediated immune mechanisms in schizophrenia.

In the present invention, the AT1 receptor was identified as an autoantigen using a novel approach. Serum samples were obtained from forty patients diagnosed with schizophrenia and forty control subject matched for age and sex. As an initial step, proteins were isolated from several different regions of postmortem human brains. Glycoproteins were then isolated from the human brain tissue and used to screen for reactivity with the sera of the schizophrenic patients and the controls by Western Blot analysis. A distinctive protein band was recognized by greater than 70% of the shizophrenic serum samples, while the same band was recognized only weakly or not at all by the control samples.

The protein with which the patient sera reacted was found to have a molecular weight of 62-64 kDa by SDS-PAGE. This protein was also found in rat brains and was not a blood component. Since there are a large number of potential candidates in this molecular weight range, it was decided to focus on glycoproteins that relate to biological indicators associated with schizophrenia rather than looking at all 64 kDa antigens. Because a characteristic symptom in a large number of schizophrenics is increased drinking called polydipsia, because the angiotensin AT1 receptor is expressed in the brain and is known to be involved in causing drinking behavior and because the AT1 receptor has a characteristic molecular weight of 64 kDa it was decided to screen the serum samples for binding to this protein. First, in order to ensure that the AT1 receptor was expressed in the brain and included in the proteins separated by SDS-PAGE, commercially available anti-AT1 antibodies were tested for their ability to react with the same protein as the patient sera and found to be positive. Then cell lines expressing the AT1 receptor were obtained and proteins isolated from these cell lines were tested for their reactivity with patient sera and it was found that the patient sera recognized the AT1 receptor protein that is expressed in high levels from these cell lines. Further confirmation of the specificity of autoantibodies was determined by using commercially available antibodies to immunoaffinity purify the glycoprotein from the cell lines. The patient sera reacted with the protein that had been immunoaffinity purified using the commercially available anti-AT1 receptor antibodies. These experiments confirm that over 70% of schizophrenic patients have autoantibodies that react with the AT1 receptor. The detection of such autoantibodies can be used as an indicator for schizophrenia.

The experimental data presented herein and detailed below demonstrate that autoantibodies to the AT1 receptor are found in schizophrenic patients and only in very low levels or not at all in controls. The detection of autoantibodies to AT1 receptor can be used to diagnose schizophrenia, to determine the severity of disease and to predict disease onset. Identification of the autoantigen recognized by the schizophrenic patients allows for the development of diagnostic kits and potential therapeutic treatments.

### EXAMPLES

The above disclosure generally describes the present invention. A more complete understanding can be obtained by reference to the following specific examples. These examples are described solely for the purposes of demonstration and or not intended to limit the scope of the invention. Changes in form and substitution of equivalents are contemplated as circumstances may suggest or render expedient.

### Example 1

### Autoantibodies

Serum samples were obtained from 40 schizophrenic patients and 40 age and sex matched controls. The serum fractions were obtained from Foothills Hospital, Calgary, Alberta using standard techniques.

### Example 2

### Preparation of Brain Antigens

Glycoproteins from human brain were obtained according to the following protocol.
1. Harvest rat/human hippocampus or other brain tissue and freeze immediately on dry ice.
2. Prepare Con-A-Sepharose beads:
   A. Wash 2.0 ml of beads with
      10 mM MgCi₂/10 mM MnCl₂ (50 ml)
      5% methyl-2-D-gucopyranoside in dH₂O (50 ml)
   0.1% SDS in 0.1 M Na Phosphate buffer pH 7.0 (50 ml)
   B. Resuspend@1:1 (V/v) in 0.1% (W/v) SDS in 0.1 M Na Phosphate buffer
3. Homogenize tissue in 0.32 M sucrose with
   0.1 mM sodium orthovanadate
   0.1 mM phenylmethylsulfonylfluoride (PMSF)
   5 μg antipain
   5 μg aprotinin
   5 μg leupeptin
   Use 250 ml of solution and sonicate on ice.
4. Quantitate sample using a spectrophotometer
5. Add enough SDS to bring the concentration up to 1% SDS and boil 5 min.
   (12.5 ml 20% SDS in 250 ml)
6. Add dH₂O to return concentration to 0.1% SDS (˜1800 ml)
7. Spin the sample to pelet tissue remains and discard. (10,000 for 10 min 4° C.)
8. Add 800 ml. of Con-A-Sepharose beads to each tube.
9. Incubate with shaking@RT for 2h.
10. Wash 6×5 min with 0.1% SDS in 0.1M Na Phosphate buffer pH 7.0
   Wash 7×5 min in 0.1M Na Phosphate buffer pH 7.0 (no SDS) (spin each time 2000 1 min)
11. Add 150 ml of: 1% SDS and boil 5 min
   4M urea (elutes glycoproteins)
   1% B-mercaptoethanol
   10% glycerol
12. To Collect Glycoproteins:
   Pierce the top, then the bottom of each microcentrifuge tube with a 30 gauge needle
   Place the pierced tube inside another tube
   Place the double tube inside a 50 ml falcon tube and spin@1000 rpm for 30 sec.

### Example 3

### Western Blot Analysis

The glycoproteins prepared according to Example 2 were separated by SDS-polyacrylamide gel electrophoresis using 7.5% polyacrylamide gels. Western blots were performed according to the following protocol:
1. 7.5% gel—run proteins@85 mA ˜2hrs
2. Transfer onto nitrocellulose or PVDF membrane overnight@30V or 1 hour@100V
3. Block 2 h 5% milk in TTBS Room temp.
4. Incubate with 1° antibody (serum diluted 1:100 or 1:200) one hour room temp.
5. Wash 3×5′ TTBS
6. Incubate with 20 reagent one hour RT
7. Wash 4×5′ TTBS
8. Incubate with substrate 5′
9. Rinse quickly in ddH₂O
10. Wrap in Saran wrap and expose to film.

The following reagents were used:

| | | | |
|---|---|---|---|
| TBS - | Tris 10 mM | Alternate Block - | 0.1% Tween 20 |
| | NaCl 140 mM | | 0.5% NP-40 |
| | pH to 7.4 | | *3.0% BSA in PBS |
| | ddH₂O to 1 L | | *fatty acid free BSA |
| TTBS - | TBS 1 L | Substrate - | Lumilight plus |
| | Tween 20 1 ml | | (Roche) |

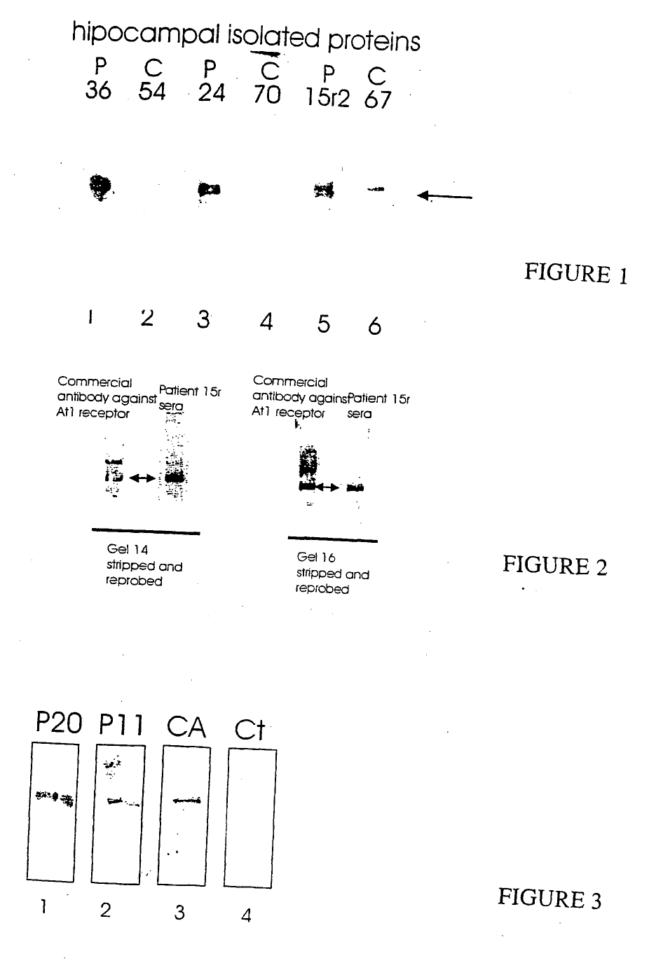

The majority of patients with schizophrenia, greater than 70%, have antibodies that recognize a protein having a molecular weight of 64 kDa which is found in the hippocampus of the human brain. The control sera reacted with the same protein only faintly or not at all. FIG. 1 illustrates the binding of three patient (P) and three control (C) samples.

### Example 4

### Identification of the Autoantigen

Three different methodologies were undertaken to confirm the identity of the autoantigen recognized by the schizophrenic patient sera.
i) Commercially available anti-AT1 receptor antibodies were obtained from Biogenesis (polyclonal rabbit antisera, catalogue no. 0560-1007) or from Santa Cruz (rabbit polyclonal. antisera no. sc-579). Brain proteins were separated by SDS-PAGE and transferred to nitrocellulose as described in Example 2. The filters were incubated with a) sc-579 at 1:5000 dilution followed by goat anti-rabbit antisera at a 1:5000 dilution or b) human sera at 1:300 dilution followed by goat anti-human immunoglobululin at 1:20000. As illustrated in FIG. 2, the comercially available antibody reacts with the same protein band as the schizophrenic patient sera.
ii) Cell lines that express high levels of AT1 receptor were obtained from cell line bank at university of Calgary. We used a hepatoma cell line huh7. Proteins were isolated from one of these cell lines, a hepatoma cell line huh7, and run on SDS-PAGE, transferred to nitrocellulose and blotted with patient or control sera. The patient sera and the control sera recognized the same protein by Western blot analysis.
iii) Commercial antibodies (Biogenesis rabbit polyclonal IgG #0560-1007) were used to immunopurify AT1 receptor from the cell lines. The rabbit IgG was used ona CNBR-sepharose column and AT1 receptors were eluted from the column, subjected to SDS-PAGE (10% gel) and transferred to PVDF membranes. As shown in FIG. 3, only the patient samples (P20 and P11) and not the control serum (Ct) reacted with the pruified protein. As a control, the commercial antibody (CA) was reacted with membrane and showed positive binding to the same band as that recognized by the patient sera)

The three assays described above indicate that a high proportion of patients with schizophrenia have autoantibodies that recognize AT1 receptors in the brain.

Although preferred aspects of the invention have been described, it is understood that variations made be made thereto without departing from the spirit of the invention.

References

Andrews P I and McNamara J O (1996). Rasmussen's encephalitis: an autoimmune disorder? Curr Opin Neurobiol 6: 673-8.
Baekkeskov S, et al. (1990). Identification of the 64K autoantigen in insulin-dependent diabetes as the GABA-synthesizing enzyme glutamic acid decarboxylase [published erratum appears in Nature Oct. 25, 1990;347(6295):782]. Nature 347: 151-6.
Ganguli R, Brar J S, Chengappa K N, Yang Z W, Nimgaonkar V L and Rabin B S (1993). Autoimmunity in schizophrenia: a review of recent findings. Ann Med 25: 489-96.
Ganguli R, Brar J S and Rabin B S (1994). Immune abnormalities in schizophrenia: evidence for the autoimmune hypothesis. Harv Rev Psychiatry 2: 70-83.
Henneberg A E, Horter S and Ruffert S (1994). Increased prevalence of antibrain antibodies in the sera from schizophrenic patients. Schizophr Res 14: 15-22.
Kilidireas K, Latov N, Strauss D H, Gorig A D, Hashirn G A, Gorman J M and Sadiq S A (1992). Antibodies to the human 60 kDa heat-shock protein in patients with schizophrenia. Lancet 340: 569-72.
Kirch D G (1993). Infection and autoimmunity as etiologic factors in schizophrenia: a review and reappraisal. Schizophr Bull 19: 355-70.
Rogers S W, Andrews P I, Gahring L C, Whisenand T, Cauley K, Crain B, Hughes T E, Heinemann S F and McNamara J O (1994). Autoantibodies to glutamate receptor GluR3 in Rasmussen's encephalitis. Science 265: 648-51.
Schwarz M J, Riedel M, Gruber R, Ackenheil M and Muller N (1999). Antibodies to heat shock proteins in schizophrenic patients: implications for the mechanism of the disease. Am J Psychiatry 156: 1103-4.

1. A method for detecting schizophrenia, or the propensity for developing shizophrenia in a person, said method comprising detecting the presence or absence of a predetermined level of antibody specific for the angiotensin AT1 receptor.

2. A method for detecting schizophrenia, or the propensity for developing shizophrenia in a person, said method comprising:
i) contacting a sample of serum from the person with an antigen comprising the AT1 receptor or a fragment thereof; and
ii) detecting binding of antibodies in said sample to said antigen;
wherein the presence of said binding indicates that said person may have or may be at risk of developing schizophrenia.

3. A peptide, comprising all or part of the amino acid sequence of the AT1 receptor, which is recognized by antibodies obtained from schizophrenic patients and recognized only weakly or not at all by control subjects.

4. Use of a peptide according to claim 2 in a kit for the diagnosis of schizophrenia.

5. A kit for the diagnosis of schizophrenia comprising:
i) the AT1 receptor or a fragment thereof;
ii) positive and negative controls for reactivity to said receptor; and
iii) reagent means for detecting binding of antibody to said receptor.

6. An assay for the diagnosis of schizophrenia in an individual comprising the steps:
A. obtaining a sample from said individual,
B. contacting said sample with an antigen comprising the AT1 receptor or a fragment thereof,
C. determining the level of binding of antibodies in said sample to said antigen, wherein a level of binding higher than a binding level of a sample from a control is indicative of schizophrenia.
